(19) United States
(12) Patent Application Publication
(10) Pub. No.: US 2021/0204503 A1
(43) Pub. Date: Jul. 8, 2021

(54) MEDICINAL CANNABIS

(71) Applicant: Agriculture Victoria Services Pty Ltd, Attwood, Victoria (AU)

(72) Inventors: Noel COGAN, Macleod, Victoria (AU); Simone Jane ROCHFORT, Reservoir, Victoria (AU); German Carlos SPANGENBERG, Bundoora, Victoria (AU); Larry Stephen JEWELL, Macleod, Victoria (AU)

(21) Appl. No.: 16/635,967

(22) PCT Filed: Aug. 1, 2018
(86) PCT No.: PCT/AU2018/050803

§ 371 Date: Jan. 31, 2020

(30) Foreign Application Priority Data
Aug. 1, 2017 (AU) 2017903047

(51) Int. Cl. A01H 6/28; A61K 36/185; A01H 1/04; A01H 5/10

(52) U.S. Cl. CPC A01H 6/28; A61K 36/185; A61K 45/06; A01H 5/10; A01H 1/04

(57) ABSTRACT

The present invention relates to medicinal cannabis plants, and cannabis plant-derived products. In particular, the present invention relates to medicinal cannabis plants having a desired cannabinoid content, methods of selecting cannabis plants having a desired cannabinoid content, chemotype and/or sex, extraction therefrom, and uses thereof. The present invention also relates to genetic markers for identifying and selecting cannabis plants having a desired chemotype and/or sex and uses thereof.

### REFERENCE TO SEQUENCE LISTING

A Sequence Listing submitted as an ASCII text file via EFS-Web is hereby incorporated by reference in accordance with 35 U.S.C. § 1.52(e). The name of the ASCII text file for the Sequence Listing is 2020-08-15_Substitute sequence listing_DAVIE70.002APC.TXT, the date of creation of the ASCII text file is Aug. 15, 2020, and the size of the ASCII text file is 100.4 KB.

### FIELD OF THE INVENTION

The present invention relates to medicinal cannabis plants, and cannabis plant-derived products. In particular, the present invention relates to medicinal cannabis plants having a desired cannabinoid content, methods of selecting cannabis plants having a desired cannabinoid content, chemotype and/or sex, extraction therefrom, and uses thereof. The present invention also relates to genetic markers for identifying and selecting cannabis plants having a desired chemotype and/or sex and uses thereof.

### BACKGROUND OF THE INVENTION

The Cannabis plant is an erect annual herb with a dioecious breeding system. Wild and cultivated forms of cannabis are morphologically variable. Presently, it is believed that there are three distinct species in the genus, but the taxonomy remains unclear: Cannabis sativa, Cannabis indica and Cannabis ruderalis. Cannabis sativa is the most commonly known.

Cannabis has a diploid genome (2n=20) with a karyotype composed of nine autosomes and a pair of sex chromosomes (X and Y). Female plants are homogametic (XX) and males are heterogametic (XY) with sex determination controlled by an x-to-autosome balance system. The estimates size of the haploid genome is 818 Mb for female plants and 843 Mb for male plants, owing to the larger size of the Y chromosome.

The cannabis plant (also referred to as marijuana, hemp) has been used for its medicinal and psychoactive properties for centuries. Currently, cannabis and its derivatives such as hashish are the most widely consumed illicit drugs in the world. Hemp forms of the cannabis plants are also used as an agricultural crop for example as a source of fibre. Cannabis use is also increasingly recognized in the treatment of a range of conditions such as epilepsy, multiple sclerosis and conditions with chronic pain.

The unique pharmacological properties of cannabis are mostly due to the presence of naturally occurring compounds known as cannabinoids. Marijuana plants have a high-THCA/low-CBDA chemotype. Hemp plants have a low-THCA/high-CBDA chemotype. There are also large differences in the specific spectrum of minor cannabinoid within these basic chemotypes.

Cannabinoids mainly accumulate in the female flowers or “buds” of the plant. Cannabinoids are also present in natural extracts derived from cannabis plants.

Tetrahydrocannabinol (THC) and cannabidiol (CBD) have been the best characterised cannabinoids to date. THC is the main psychoactive cannabinoid and the compound responsible for the analgesic, antiemetic and appetite-stimulating effects of cannabis. Non-psychoactive cannabinoids such as cannabidiol (CBD), cannabichromene (CBC) and tetra-hydrocannabivarin (THCV), which possess diverse pharmacological activities, are also present in some strains.

Pharmaceutical compositions comprising cannabinoids having specific ratios of CBD to THC are useful in the treatment and management of specific diseases or medical conditions. For example, a pharmaceutical composition containing a high ratio of CBD compared to THC is useful in the field of epilepsy. Conversely, a pharmaceutical composition containing a high ratio of THC compared to CBD is useful in the field of pain relief.

The amount of particular components in the cannabis plant or extracts therefrom may impact the efficacy of therapy and potential side effects. Accordingly, cannabis plant varieties having specific therapeutic component profiles may be useful in the production of pharmaceutical compositions for the treatment of specific conditions.

Current methods for the determination of amounts of cannabinoids in a cannabis plant or extracts therefrom have limitations around resolution sensitivity, reliability and throughput.

There exists a need to overcome, or at least alleviate, one or more of the difficulties or deficiencies associated with the prior art.

### SUMMARY OF THE INVENTION

In one aspect, the present invention provides a method of identifying a cannabis plant having high THC content and/or high CBD content, wherein the method includes detecting a genetic variation associated with the THCAS gene and/or CBDAS gene in the cannabis plant.

In a preferred embodiment, the method may further include correlating said genetic variation with high THC content and/or high CBD content.

All Cannabinoids, including THC and CBD are derived from the precursor cannabigerolic acid (CBGA).

Several key enzymes have been identified in the cannabinoid pathway that dictate whether the CBGA is converted to cannabidiolic acid (CBDA), tetrahydrocannabinolic acid (THCA) or less commonly, remain as cannabigerolic acid (CBGA) or become cannabichromene acid (CBCA). Decarboxylation then converts THCA into THC, CBDA into CBD and CBCA into CBC. It is in this form that the cannabinoids are generally used for medicinal purposes.

The main two oxidocyclases, THCA synthase (THCAS) and cannabidiolic acid synthase (CBDAS) are involved in the conversion of the CBGA precursor to THCA and CBDA respectively. Therefore, the amount of THCAS versus CBDAS present in a cannabinoid plant can determine the amount each different cannabinoid in a specific cannabis plant. This is also referred to as a THCAS:CBDAS ratio.

Determining the presence or absence of one or more variations of genetic markers associated with the THCAS and/or CBDAS genes in a cannabis plant may be used to identify the relative THCAS and/or CBDCAS that is expressed and the THC/CBD content (or THC/CBD chemotype) in the cannabis plant. The genetic variations are therefore useful in a method to determine the THC/CBD chemotype of a cannabis plant. Additionally, the genetic markers may be used as an effective tool to screen the THC/CBD content at the genetic level. Furthermore, the genetic markers may be used in the application of genome editing to optimise THC/CBD chemotype in a cannabis plant.

The cannabis plant can be selected from the following species (or sub-species) Cannabis sativa, Cannabis indica, Cannabis ruderalis, or hybrid thereof, preferably the cannabis plant is Cannabis sativa.

The term “cannabinoids” as used herein refers to a class of compounds that act on the cannabinoid receptors. Cannabinoids found in the cannabis plants include, without limitation: cannabigerol (CBG), cannabichromene (CBC), cannabidiol (CBD), tetrandrocannabinol (THC), cannabinol (CBN), cannabinodiol (CBDL), cannabicyclol (CBL), tetrahydrocannabivarin (THCV), cannabidivarin (CBDV), cannabichromevarian (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), d9-THC, exo-THC. 11-OH-d9-THC, 11-nor-d9-THC, d9-THCA-A, d8-THC12

“Terpenes” or “terpenoids” refer to a class of chemicals produced by plants, including cannabis. These compounds are often aromatic hydrocarbons and have strong aroma associated with them. Terpenes known to be produced by cannabis include, without limitation, aromadendrene, bergamottin, bergamotol, bisabolene, borneol, alpha-3-carene, caryophyllene, cinole/eucalyptol, p-cymene, dihyrojasmne, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpinol, terpineol-4-ol, terpinolene, and derivatives, isomers, enantiomers thereof.

The term “high THC content” as used herein refers to the content by weight of cannabinoid THC in an extract that is derived from the cannabis plant which is higher than the CBD content by weight. The ratio by weight of THC to CBD may be more than 1, preferably more than about 1.2, more preferably more than about 1.5, more preferably more than about 2. Preferably the ratio by weight of THC to CBD is between about 400:1 and 2:1, preferably about 100:1 to 2:1, more preferably about 50:1 to 2:1, more preferably about 25:1 to 2:1, more preferably about 10:1 to 2:1, more preferably about 5:1 to 2:1. In some instances “high THC content” may refer to a cannabis plant which does not have any CBD content.

The term “high CBD content” as used herein refers to the content by weight of cannabinoid CBD in an extract that is derived from the cannabis plant which is higher than the THC content by weight. The ratio by weight of CBD to THC may be more than 1, preferably more than about 1.2, more preferably more than about 1.5, more preferably more than about 2. Preferably the ratio by weight of CBD to THC is between about 400:1 to 2:1, preferably about 100:1 to 2:1, more preferably about 50:1 to 2:1, more preferably about 10:1 to 2:1, more preferably about 5:1 to 2:1. In some instances “high CBD content” may refer to a cannabis plant which does not have any THC content.

The term “chemotype” as used herein is meant to refer to the content of chemical compounds found in the cannabis plant. This includes, but not limited to the presence and/or absence of specific cannabinoids found in an extract of the cannabis plant. For example, the CBD/THC chemotype as used herein refers to the CBD and/or THC content found in the cannabis plant. This also includes the presence or absence of other compounds, including cannabinoids in addition to or other than THC/CBD, and terpenes or terpinoids.

Accordingly, in a further aspect of the invention, the cannabis plant further includes one or more cannabinoids selected from the group consisting of: cannabigerol (CBG), cannabichromene (CBC), cannabidiol (CBD), tetrandrocannabinol (THC), cannabinol (CBN), cannabinodiol (CBDL), cannabicyclol (CBL), tetrahydrocannabivarin (THCV), cannabidivarin (CBDV), cannabichromevarian (CBCV), cannabigerovarin (CBGV), cannabigerol monomethyl ether (CBGM), cannabinerolic acid, cannabidiolic acid(CBDA), cannabinol propyl variant (CBNV), cannabitriol (CBO), tetrahydrocannabinolic acid (THCA), tetrahydrocannabivarinic acid (THCVA), d9-THC, exo-THC. 11-OH-d9-THC, 11-nor-d9-THC, d9-THCA-A, d8-THC12.

Accordingly, in a further aspect of the invention, the cannabis plant further includes terpenes. Preferably, the terpenes are selected from one or more of the following group: aromadendrene, bergamottin, bergamotol, bisabolene, borneol, alpha-3-carene, caryophyllene, cinole/eucalyptol, p-cymene, dihydrojasmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpinol, terpineol-4-ol, terpinolene, and derivatives, isomers, enantiomers thereof.

The term “genetic variation” as used herein is meant to refer to a change of the DNA, RNA and/or protein sequence. The genetic variation may be, but is not limited to, a single polynucleotide change in the DNA sequence. The genetic variation may also result in other changes in the protein expression level, including premature stop codons that result in truncated proteins. The function of the resulting protein that is expressed may or not be affected.

The genetic variation may be detected by various techniques, including detecting the presence or absence of polymorphic markers such as simple sequence repeats (SSRs) or mating type gene markers. Alternatively, or in addition, the genetic variation may be detected by sequencing genomic and/or mitochondrial DNA and/or ribosomal RNA, and performing sequence comparisons to databases of known nucleic acid sequences, for example known sequences of the THCAS and/or CBDAS genes.

The analysis of genetic variation may be performed on nucleic acid samples obtained from the cannabis plant. Preferably the nucleic acid samples may be extracted from the buds, leaves or flowers of the cannabis plant. The nucleic acid samples maybe DNA or RNA. Only small amounts are required for analysis and suitable for automation.

In one aspect of the present invention, the genetic variation is associated with the THCAS gene.

In one embodiment of this aspect of the invention, the genetic variation results in one or more amino acid changes in the expression of the THCAS gene. Preferably the genetic variation is selected from either one or both: Lys to Met at position 8190 and Leu to Phe at position 8201 in the THCAS gene. The applicant has found that the variation in the DNA sequence of the THCAS gene in either one or both of these two positions results in amino acid changes in the THCAS. Without being bound by any particular theory or mode of action, it is believed that this genetic variation may play a role in methylation patterns.

In another embodiment, the genetic variation is associated with the CBDAS gene.

Genetic variations or mutations resulting in a premature stop codon in the expression of the CBDAS gene have been identified and described in van Bakel et al (2011). The applicant has now quantified these from a pan genome evaluation of the cannabis plant.

In another aspect of the invention there is provided a cannabis plant having a high THC content and/or high CBD content. Preferably, the cannabis plant is identified according the method described herein.

In one embodiment of this aspect of the invention, there is provided a cannabis plant wherein the CBD is present in the cannabis plant in an amount by weight greater than the amount by weight of THC. In some embodiments, the cannabis plants do not have any THC.

In another embodiment of this aspect of the invention, there is provided a cannabis plant wherein the THC is present in the cannabis plant in an amount by weight greater than the amount by weight of CBD. In some embodiments, the cannabis plants do not have any CBD.

In another embodiment of this aspect of the invention, there is provided a seed, cell, part of a plant and/or a plant-derived product derived from a plant according to the present invention. A plant-derived product may be but not limited to an oil, tincture, flowers, buds and/or leaves. The flowers and/or leaves maybe dried or cured.

The cannabis plant identified according to the invention is useful in breeding cannabis strains for medicinal purposes, or medicinal cannabis. Medicinal cannabis strains are useful for the preparation of pharmaceutical composition containing the desired amount of cannabinoids, preferably medicinal cannabis strains having a high THC content and/or high CBD content.

Accordingly, in another aspect there is provided a method of breeding a cannabis plant including the step of identifying or selecting a cannabis plant having high THC content and/or high CBD content as herein described.

In a preferred embodiment, the method may further include propagating or crossing the selected plant.

In a further aspect there is provided a use of a cannabis plant having high THC content and/or high CBD content identified by the methods described herein for breeding a medicinal cannabis plant.

In another aspect of the invention there is provided a method of preparing a composition which includes the steps of:
a. providing a cannabis plant identified according to the invention; and
b. preparing an extract from the cannabis plant having high THC content and/or high CBD content.

Preferably the composition is a pharmaceutical composition. Preferably the method includes the further step of combining the extract with one or more pharmaceutical excipients.

In one preferred embodiment of this aspect of the invention, the composition further includes one or more other cannabinoids selected from: cannabigerol (CBG), cannabichromene (CBC), cannabidiol (CBD), tetrandrocannabinol (THC), cannabinol (CBN), cannabinodiol (CBDL), cannabicyclol (CBL), tetrahydrocannabivarin (THCV), cannabidivarin (CBDV), cannabichromevarian (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), d9-THC, exo-THC. 11-OH-d9-THC, 11-nor-d9-THC, d9-THCA-A, d8-THC12, preferably CBDA and THCA.

Preferably, the composition further includes one or more terpenes selected from the group consisting of aromadendrene, bergamottin, bergamotol, bisabolene, borneol, alpha-3-carene, caryophyllene, cinole/eucalyptol, p-cymene, dihydrojasmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpinol, terpineol-4-ol, terpinolene, and derivatives, isomers, enantiomers thereof.

In another preferred embodiment, the method further includes the step of heating plant material of (a) to a temperature of from about 60° C. to about 225° C., preferably about 100° C. to about 150° C., more preferably about 110° C. to 130° C., more preferably at about 120° C., to decarboxylate the acid form of any cannabinoids present in the extract.

In another preferred embodiment, the extract is prepared by at least one of the following procedures: maceration, percolation, extraction with a solvent or supercritical fluid extraction.

In another preferred embodiment of the invention the composition is further formulated into a pharmaceutical composition.

In another aspect of the invention, there is provided a pharmaceutical composition prepared by the methods described herein.

In one embodiment of this aspect, there is provided a pharmaceutical composition wherein CBD is present in an amount by weight greater than THC. In some embodiments, the composition does not contain any THC.

In another embodiment of this aspect of the invention, there is provided a pharmaceutical composition wherein the THC is present in an amount by weight greater than CBD. In some embodiments, the composition does not contain any CBD.

Preferably, the composition further includes one or more other cannabinoids selected from cannabigerol (CBG), cannabichromene (CBC), cannabidiol (CBD), tetrandrocannabinol (THC), cannabinol (CBN), cannabinodiol (CBDL), cannabicyclol (CBL), tetrahydrocannabivarin (THCV), cannabidivarin (CBDV), cannabichromevarian (CBCV), cannabigerovarin (CBGV), cannabigerol monomethyl ether (CBGM), cannabinerolic acid, cannabidiolic acid (CBDA), cannabinol propyl variant (CBNV), cannabitriol (CBO), tetrahydrocannabinolic acid (THCA), tetrahydrocannabivarinic acid (THCVA), d9-THC, exo-THC. 11-OH-d9-THC, 11-nor-d9-THC, d9-THCA-A, d8-THC12.

Preferably, the composition further includes one or more terpenes selected from the group consisting of aromadendrene, bergamottin, bergamotol, bisabolene, borneol, alpha-3-carene, caryophyllene, cinole/eucalyptol, p-cymene, dihydrojasmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpinol, terpineol-4-ol, terpinolene, and derivatives, isomers, enantiomers thereof.

In another aspect of the invention there is provided a pharmaceutical composition for use in the manufacture of a medicament for the treatment of a medical condition. Preferably the medical condition is pain relief or management thereof or epilepsy.

Alternatively, in another aspect of the invention there is provided a pharmaceutical composition for use in the manufacture of a medicament for the treatment of a therapeutic condition. Preferably the therapeutic condition is pain relief or management thereof or epilepsy.

THC has an analgesic, anti-spasmodic, anti-tremor, anti-inflammatory, appetite stimulant and anti-emetic properties whilst CBD has anti-inflammatory, anti-convulsant, anti-psychotic, anti-oxidant, neuroprotective and immunomodulatory effects.

Pharmaceutical compositions comprising cannabinoids having specific ratios of CBD to THC are useful in the treatment and management of specific diseases or medical conditions. For example, a pharmaceutical composition containing a high ratio of CBD compared to THC is useful in the field of epilepsy. Conversely, a pharmaceutical composition containing a high ratio of THC compared to CBD is useful in the field of pain relief.

According to this aspect of the invention, a composition having CBD in an amount by weight greater than the amount by weight of THC may be used in the treatment of epilepsy.

According to another aspect of the invention, a composition having THC in an amount by weight greater than the amount by weight of CBD is used in the treatment of pain and/or management thereof.

In a further aspect of the present invention there is provided use of a composition according to the present invention for the treatment of a therapeutic condition, wherein the therapeutic condition is epilepsy.

In a further aspect of the present invention there is provided a method of treating a therapeutic condition including the administration of a composition according to the present invention to a patient in need of treatment, wherein the therapeutic condition is epilepsy.

In these aspects of the present invention, preferably the CBD is present in the composition in an amount by weight greater than the amount by weight of THC.

In a further aspect of the present invention there is provided use of a composition according to the present invention for the treatment of a therapeutic condition, wherein the therapeutic condition is pain relief or management thereof.

In a further aspect of the present invention there is provided a method of treating a therapeutic condition including the administration of a composition according to the present invention to a patient in need of treatment, wherein the therapeutic condition is pain relief or management thereof.

In these aspects of the present invention, preferably the THC is present in the composition in an amount by weight greater than the amount by weight of CBD.

The present invention will now be more fully described with reference to the accompanying Examples and drawings. It should be understood, however, that the description following is illustrative only and should not be taken in any way as a restriction on the generality of the invention described above.

### DETAILED DESCRIPTION OF THE EMBODIMENTS

In the Figures:

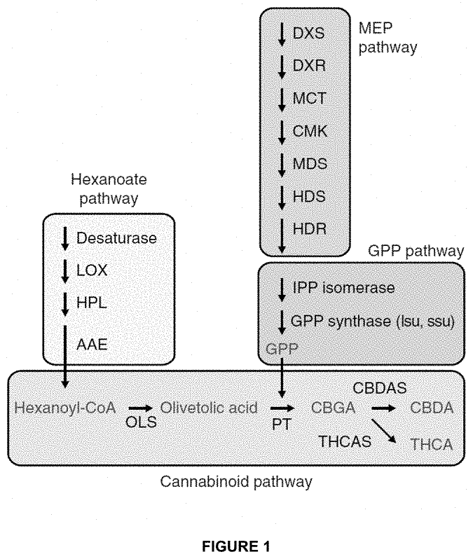

FIG. 1 shows a schematic diagram of cannabinoid pathway in a cannabis plant reproduced from van Bakel et al (2011).

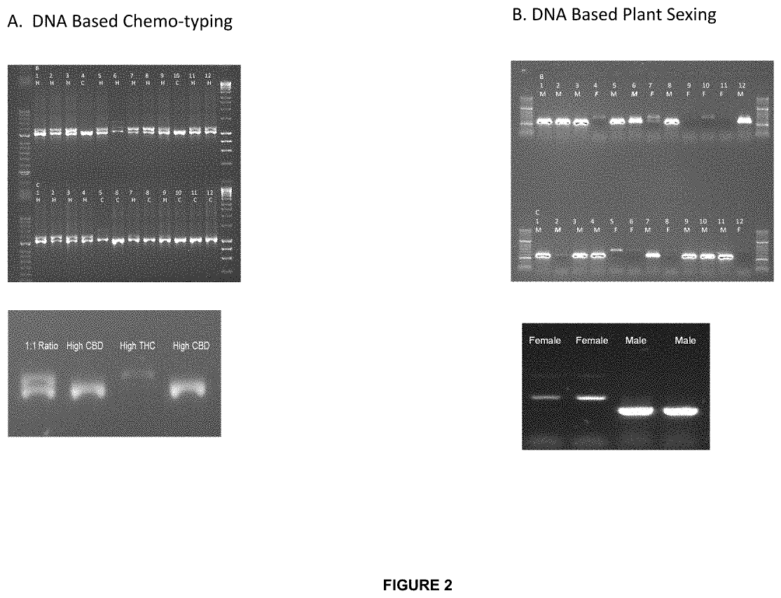

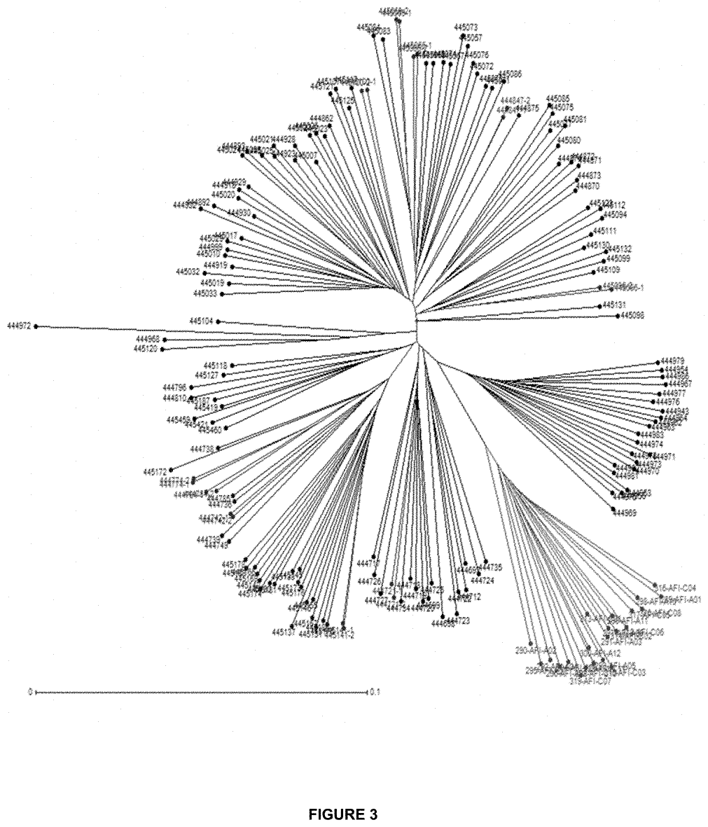

FIG. 2A shows DNA analysis of cannabinoid content in a DNA extract derived from a cannabis plant on agarose gel (i) DNA markers used to determine chemotype of cannabis plant extract (ii) detailed view of gel shown in (i).

FIG. 2B shows determination of sex in the cannabinoid plant by analysis of a DNA extract derived from a cannabis plant on an agarose gel (i) DNA markers used to determine plant sex of a cannabis plant (ii) detailed view of gel shown in (i).

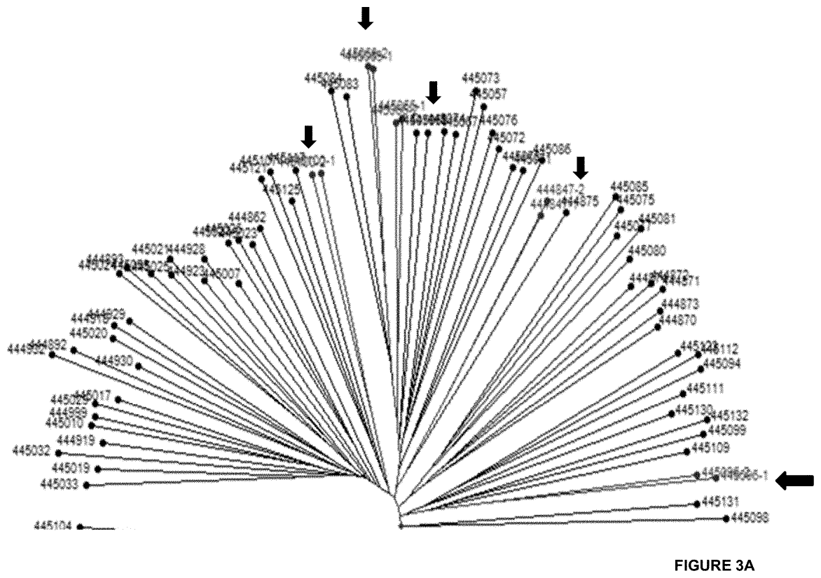

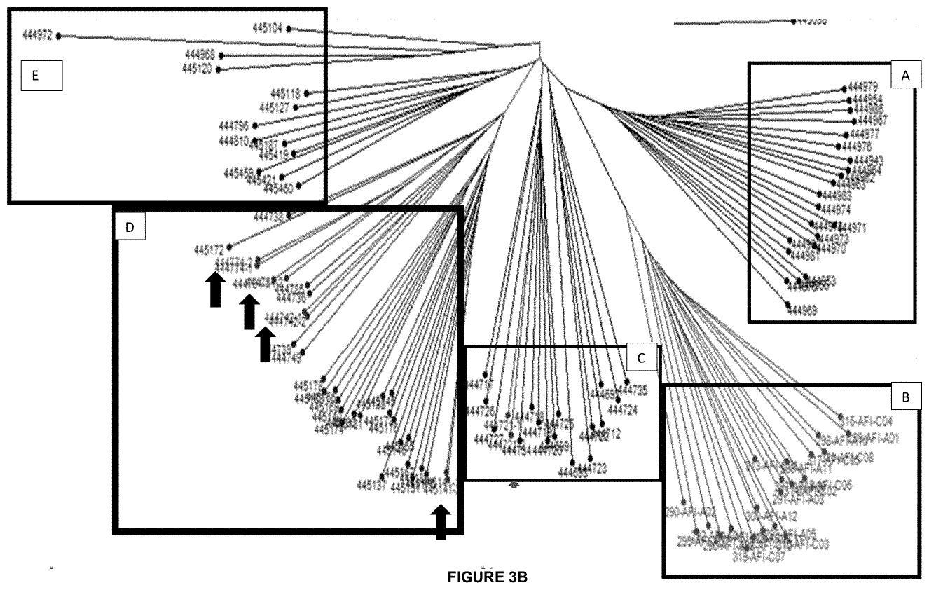

FIG. 3 shows genetic diversity of cannabis plants that have been whole genome sequenced.

FIG. 3A shows the enlarged top half section of FIG. 3. All plants in this section have high THC. Arrows denote duplicated samples.

FIG. 3B shows the enlarged bottom half section of FIG. 3. Boxes Arrows denote duplicated samples. Box B represents plants having high CBD; Box D represents plants having both CBD and THC; Boxes A, C and E represent plants with high THC; Arrows denote duplicated test samples.

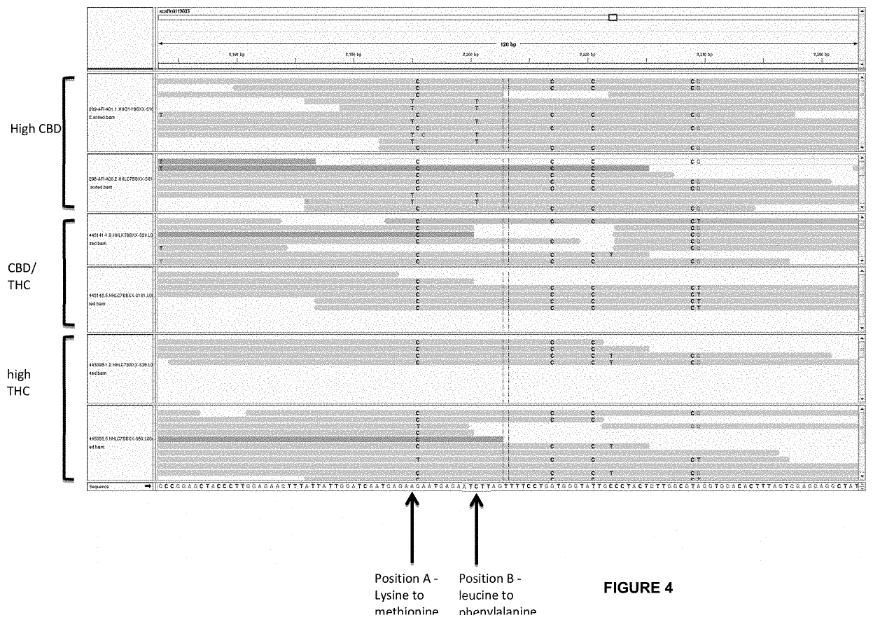

FIG. 4 shows nucleic acid changes that alter amino acid sequences in the THCAS gene scaffold 19603. Analysis of plants was performed on plants having (i) high CBD content (Rows 1 and 2); (ii) both high CBD and high THC content (rows 3 and 4); (iii) high THC (rows 5 and 6). Arrow A denotes change in nucleic acid position 8190 resulting in amino acid change Lys to Met. Arrow B denotes change in nucleic acid position 8201 resulting in amino acid change Leu to Phe. The sequence of a 120 bp fragment of the THCAS gene shown at the bottom of this figure corresponds to SEQ ID NO 3.

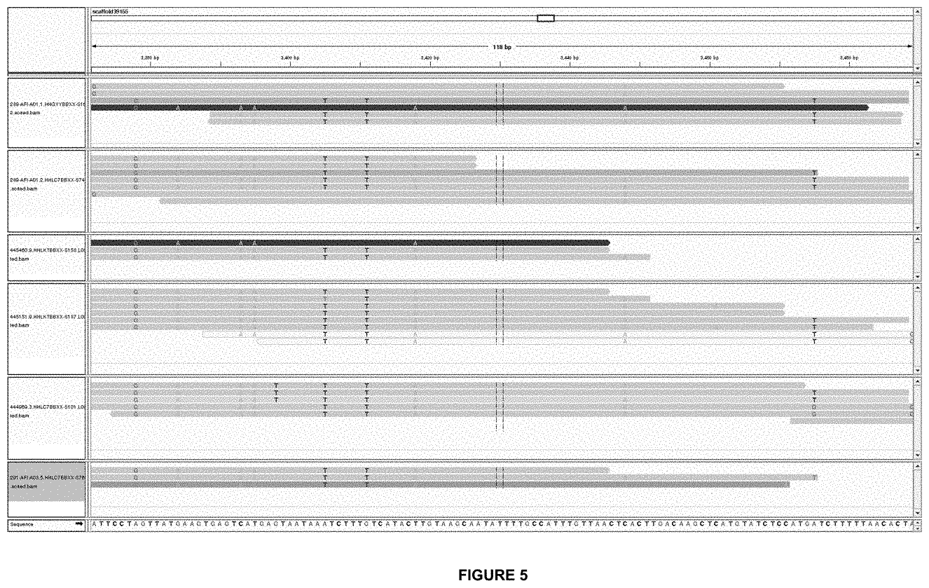

FIG. 5 shows analysis of CBDAS gene and identification of premature stop codon at position 3448. The sequence of the fragment of the CBDAS gene shown at the bottom of this figure corresponds to SEQ ID NO: 6.

FIG. 5 shows protocol for tissue culture based plant propagation from cutting to aseptic based root induction on medium. Each step is shown in order from A to H.

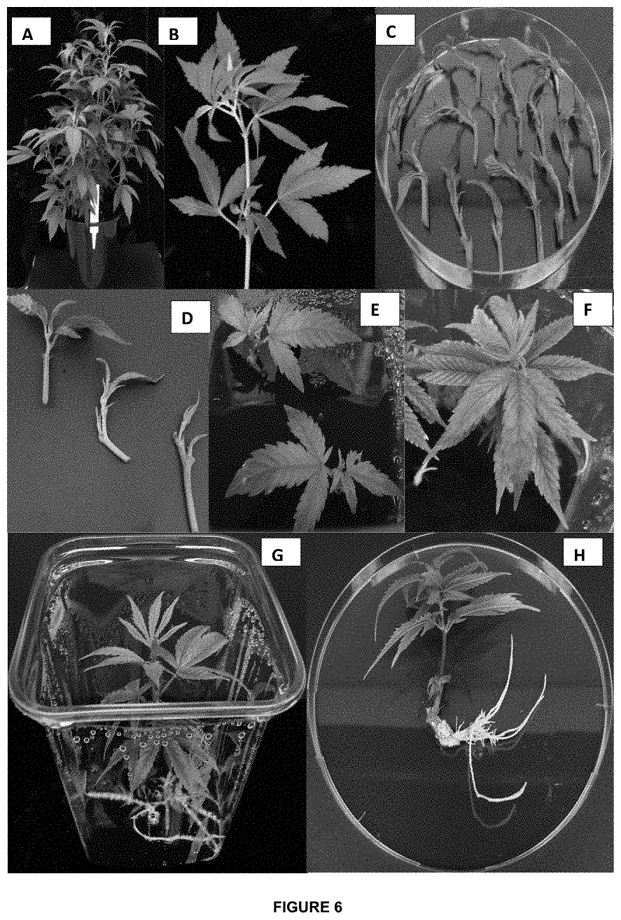

FIG. 6 shows protocol for robust production of continuous supply of young in vitro material via synthetic seed technology. Each step is shown in order from A to H.

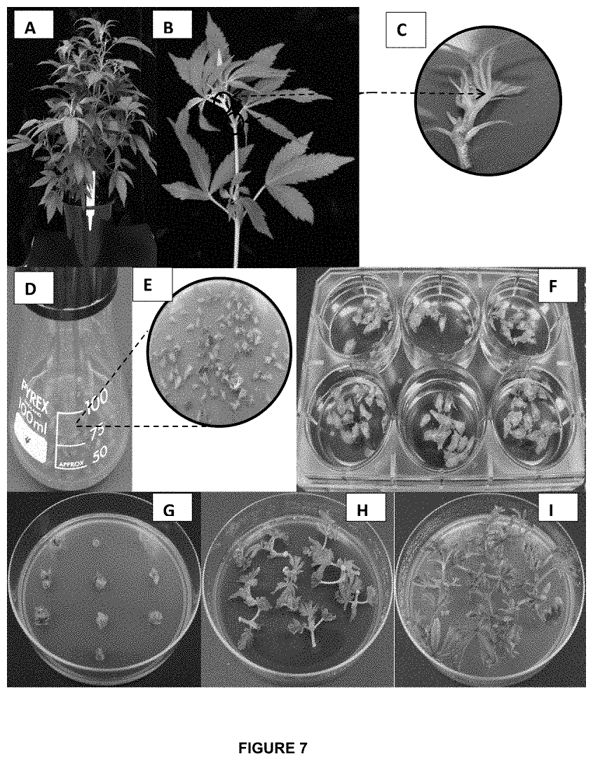

FIG. 7 shows chemical structure of cannabinoid and terpene metabolites analysed in cannabis: a-pinene, limonene, g-eudesmol, CBD, CBDA, d9-THCA-A, THC.

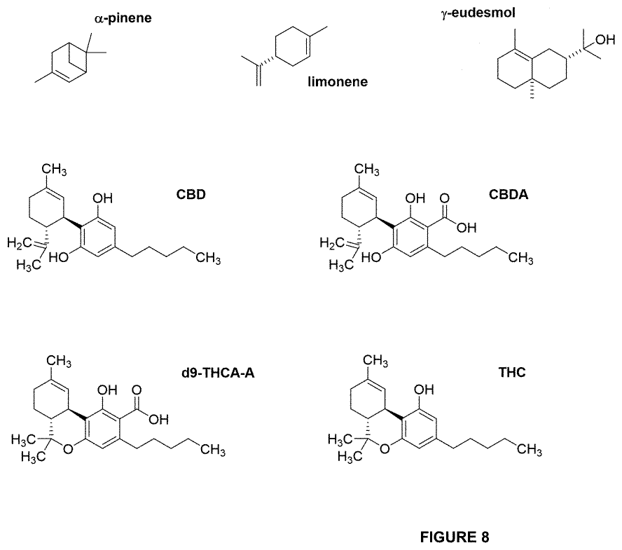

FIG. 8 shows analysis of cannabis plant material for three different medicinal cannabis strains 1, 2, 3 for volatinomics including alcohols, aldehydes, monoterpenes and sesquiterpenes by GCMS (static headspace) analysis.

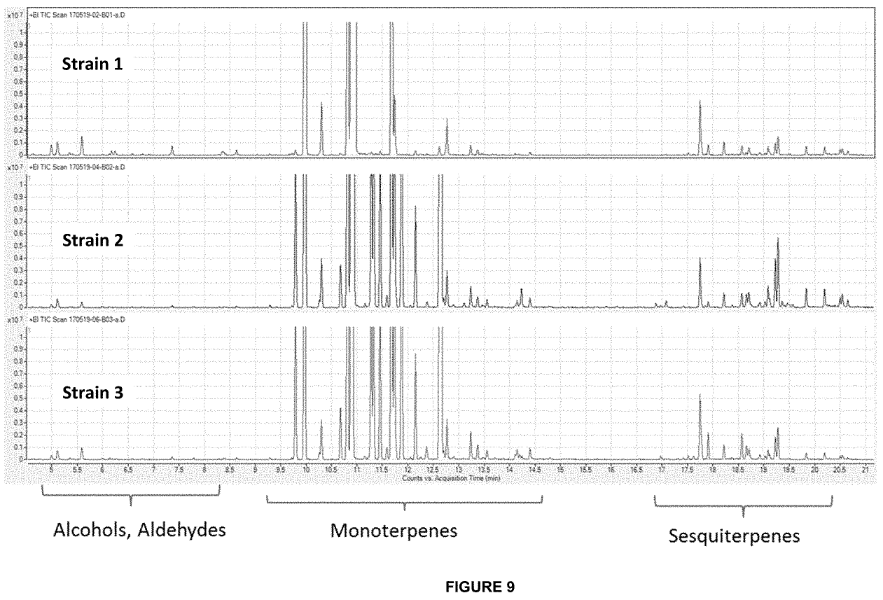

FIG. 9 shows comparison of analysis of cannabis plant material by Solid Phase Microextraction (SPME) compared to GCMS static headspace.

FIG. 10 shows analysis of monoterpenes in three different medicinal cannabis strains.

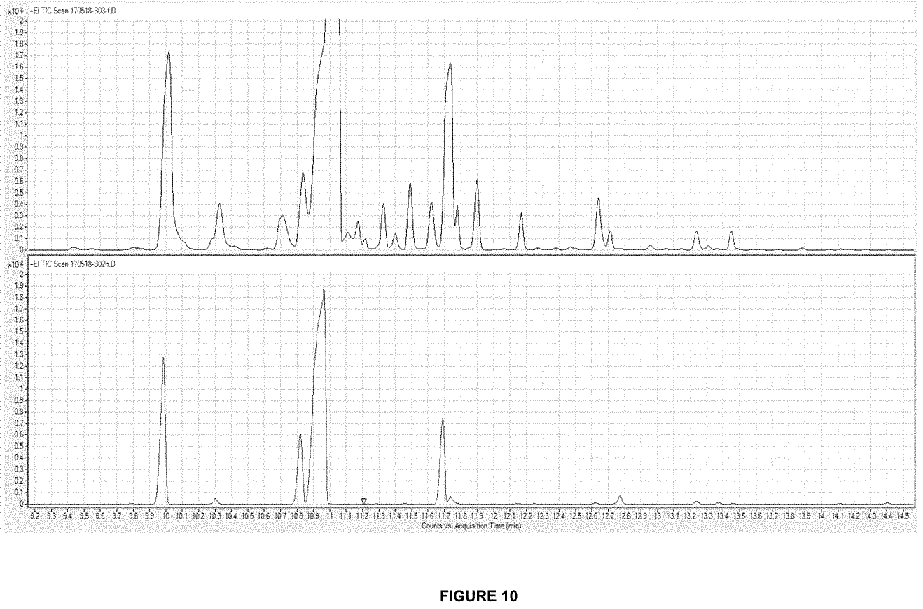

FIG. 11 shows analysis of sesquiterpenes in three different medicinal cannabis strains.

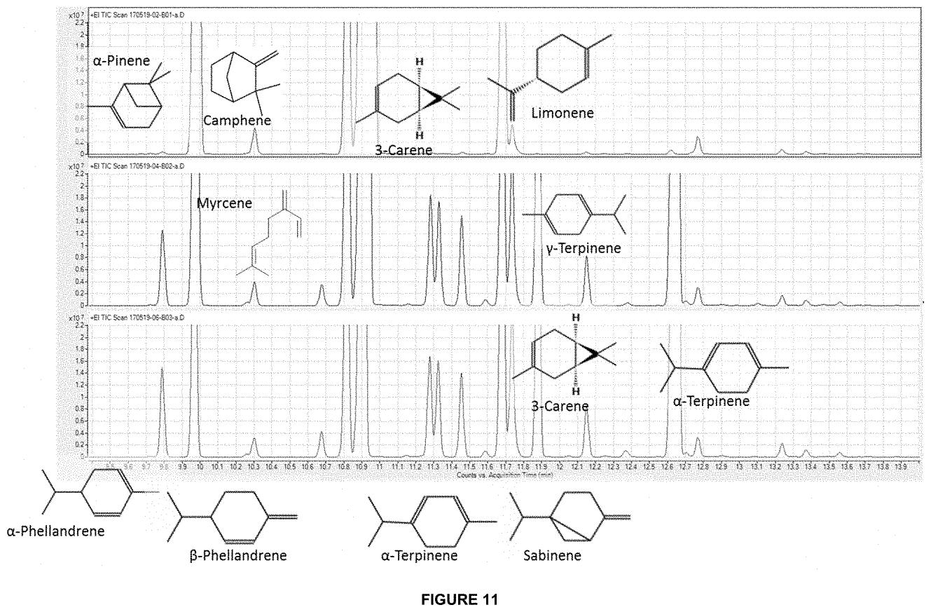

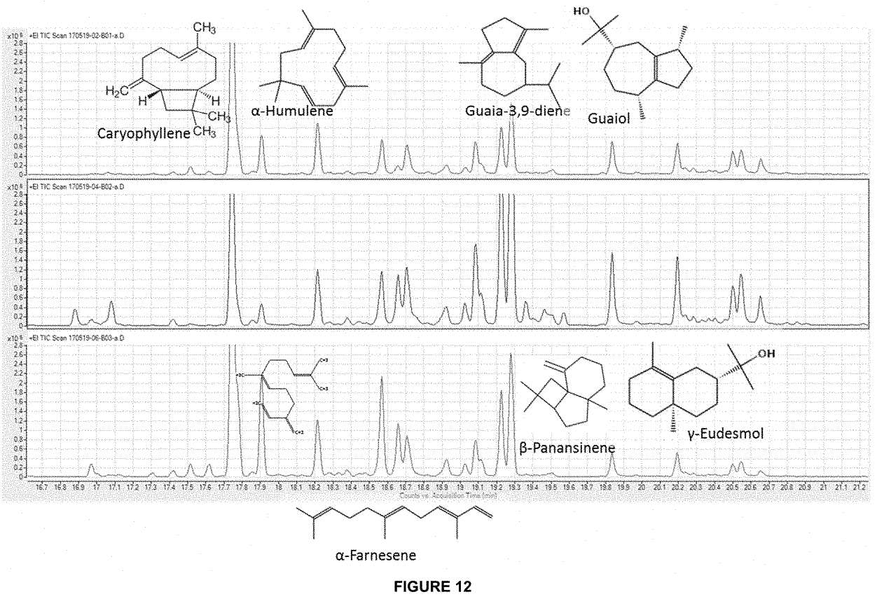

FIG. 12 shows analysis of alcohols and aldehydes in three medicinal cannabis strains.

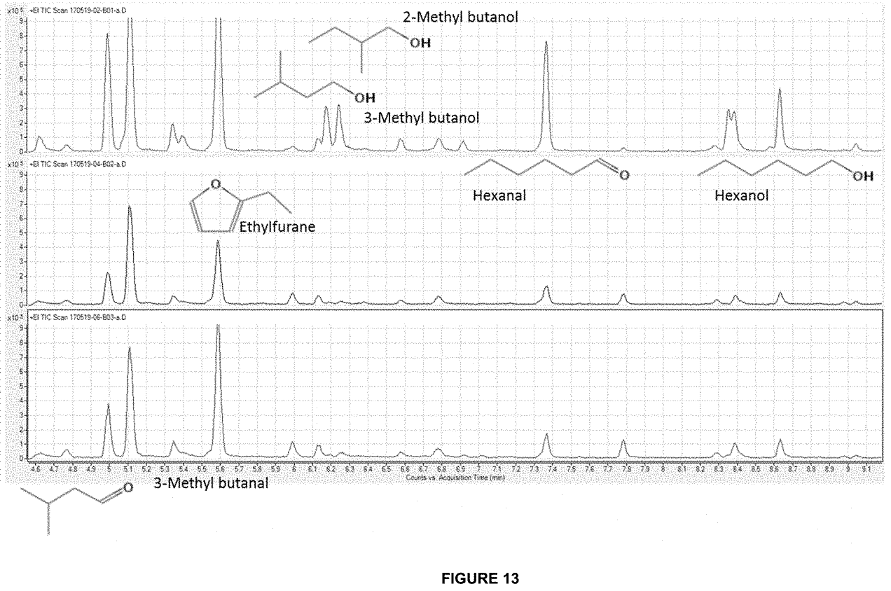

FIG. 13 shows comparison of detection of volatile material in air dried (A) versus cured (B) plant materials. Air dried materials are shown in the above line and cured plant materials are shown in the line below highlighted in box with dotted line.

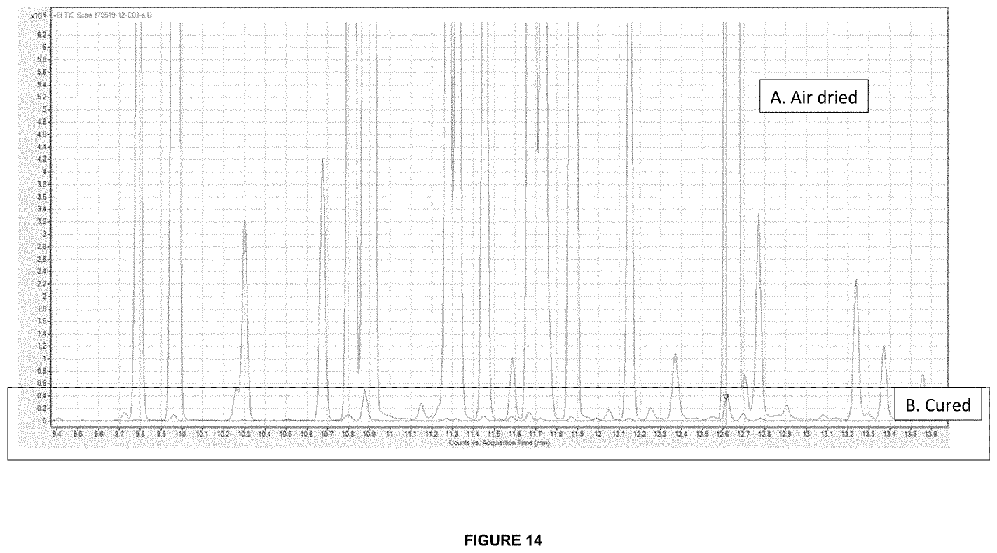

FIG. 14 shows analysis of ion extracted chromatograms of mixed standards (Top line). Line A shows peaks for CBDVA and 11-0H-d9-THC; Line B shows peaks for 11-nor-9-0H-d9-THC; Line C shows peaks for CBDV and THCV; Line D shows peaks for CBDA and d9-THCA-A; Line E shows peak for CBGA; Line F shows peak for CBG; Line G shows peaks for CBD exo-THC and d9-THC, d8-THC, CBL, CBC; Line H shows peak for CBN.

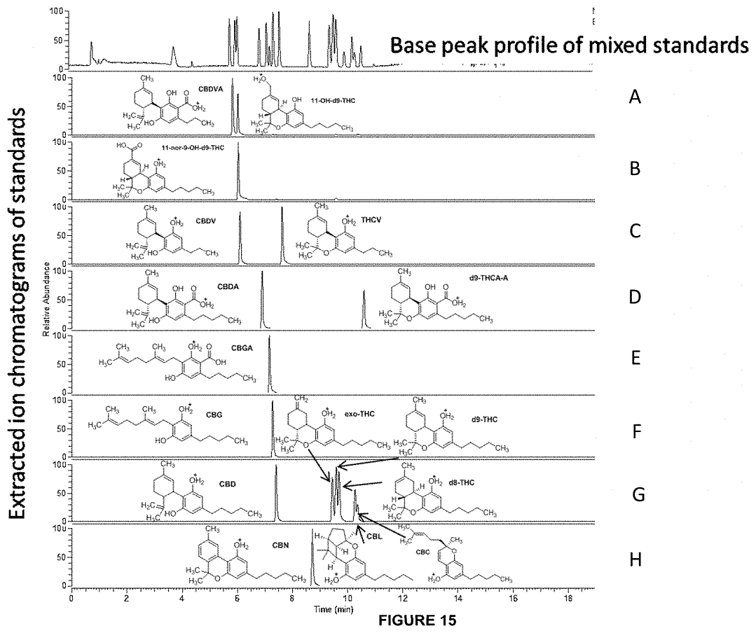

FIG. 15 shows the comparison of cannabinoid composition in (A) dried (air-dried) and (B) cured plant material extracted with methanol prior to analysis.

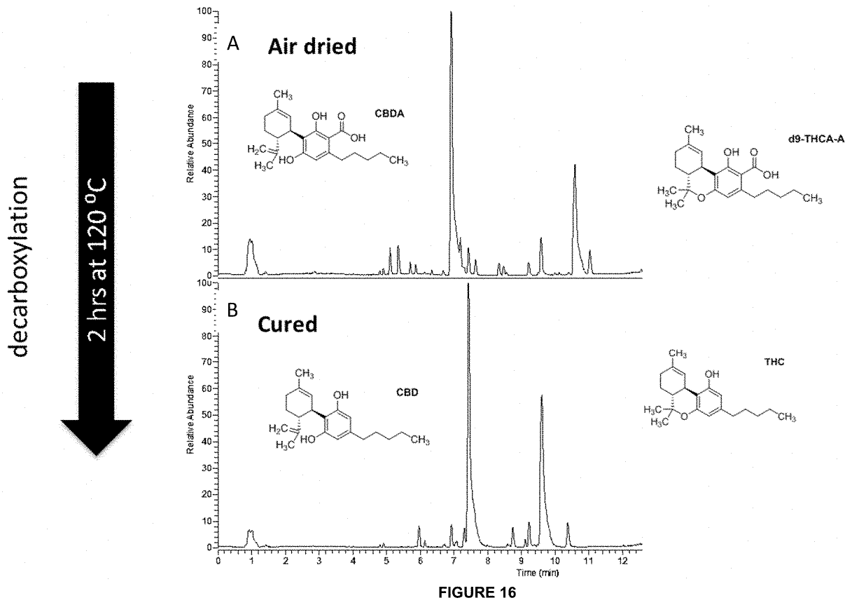

FIG. 16 shows UHPLC-PDA quantification of the main cannabinoids (CBDA, CBD, THC, THCAA) in the buds of one cannabis strain which has been sampled weekly for 6 weeks (denoted W1, W2, W3, W4, W5, W6). For each week (in order from left to right), the first bar measures CBDA; the second bar measures CBD, the third bar measures THC; the fourth bar measure THCAA.

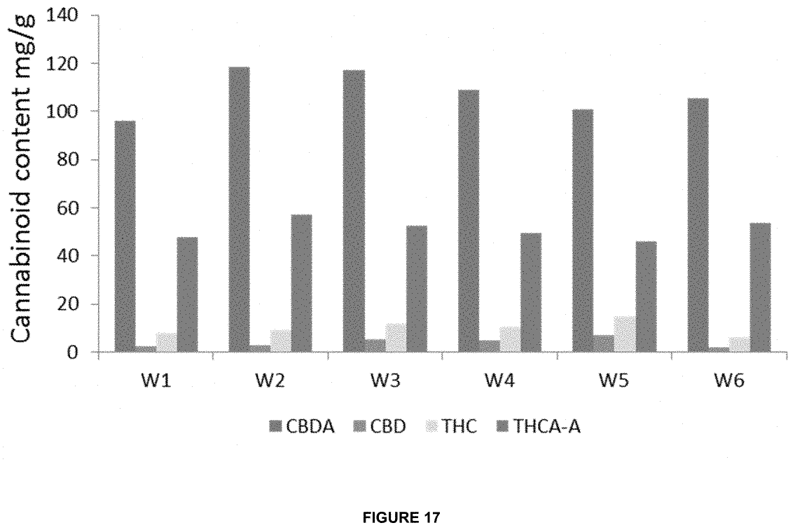

FIG. 17 shows a statistical analysis (Principal Component Analysis, PCA) of LCMS data from available cannabis strains.

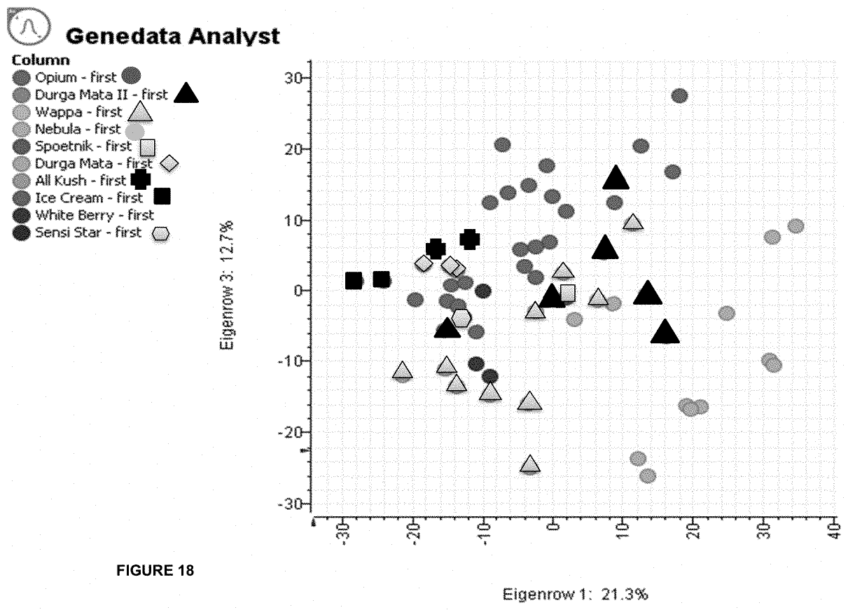

FIG. 18 shows NMR spectra for cannabinoid CBD and CBDA standards

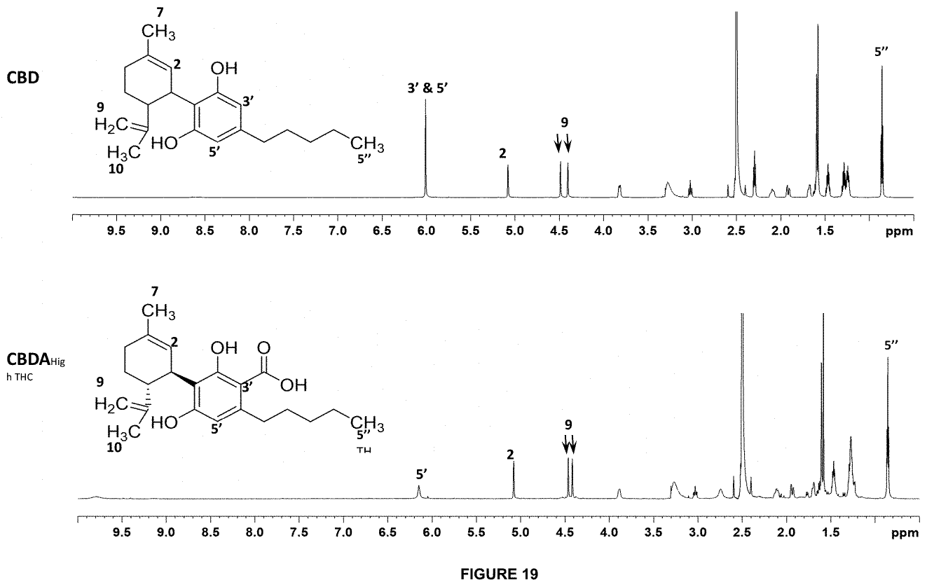

FIG. 19 shows NMR spectra for cannabinoid compound standards. In order from top to bottom: D9-THCAA, d9-THC, CBDA, CBD, and Mixture (CBD+CBDA+THC+THCAA)

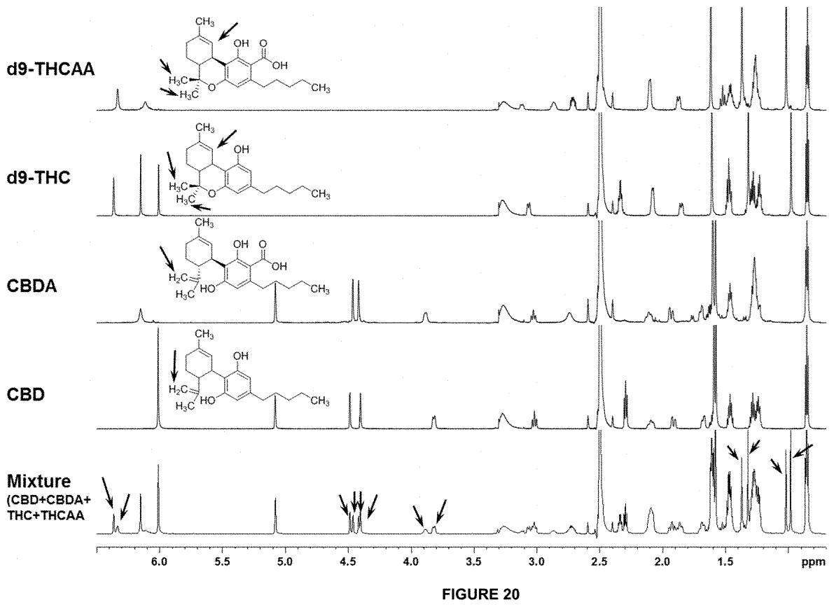

FIG. 20 shows the NMR spectra of cannabis strain. The asterisk denotes the presence of glucose metabolite in the sample.

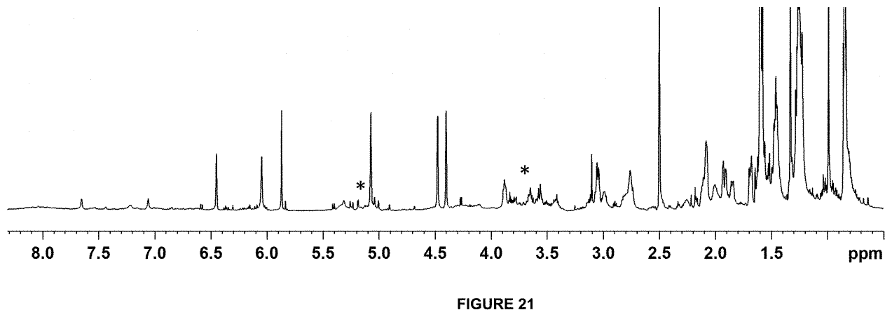

FIG. 21 shows NMR spectra of cannabis strain after (i) air drying (top line) (ii) cured compared (middle line) (iii) mixed standards (bottom line). Arrows denote peaks for CBD (arrow A), CBDA (arrow B), THC (arrow C), and CBD or CBDA (arrow D).

The invention will now be described with reference to the following non-limiting examples.

### EXAMPLE 1—CANNABINOID PATHWAY

FIG. 1 shows the cannabinoid pathway and some of the genes involved. This pathway shows that the CBG-A, or cannabigerolic acid, is the precursor compound from which THCA and CBDA are formed by the expression of the THCAS gene and CBDAS gene respectively.

### EXAMPLE 2—APPLICATION OF RUDIMENTAL DNA MARKERS IN DETERMINING CHEMOTYPE AND PLANT SEX

Assays for the determination of chemotype and plant sexing currently exist as shown in FIGS. 2A and 2B respectively.

The DNA marker assay for determining cannabinoid content was performed as described in Pacifico et al (2006). 3 PCR primer reaction amplifies a pair of products from the THCAS and CBDAS genes. The presence of the band is linked with the functional variant of the gene and therefore the assay indicates the THC/CBD chemotype of the cannabis plant.

The DNA marker assay for determining plant sex was performed as described in Mandolino et al (1999). The assay is a PCR based primer reaction—the size of the product indicates whether the plant is male or female.

There are limitations with these methods as this is based on technology with limitations around: resolution, sensitivity, reliability and throughput.

### EXAMPLE 3—WHOLE GENOME SEQUENCING OF CANNABIS STRAINS

Current genomic resources for Cannabis plants are not well described. A draft genome and transcriptome sequence of C. sativa, Purple Kush (PK) a marijuana strain that is widely used for its medicinal effects has been reported (Van Bakel et al (2011)).

Through the availability of short-read sequencing technology a cohort of around 200 medicinal cannabis plants have now been genome sequenced. The cannabis strains analysed include: Opium; Durga Mata; Durga Mata II; Wappa; Nebula; Spoetnik; Ali Kush; Ice Cream; White Berry; Sensi Star.

Genome sequencing was performed using short sequence read technology through the Illumina HiSeq300 platforms. DNA from subject plants was enzymatically sheared using the ShredF method (Shinozuka et al (2015)), synthetic DNA adaptors were then ligated and the molecules amplified and then processed on the Illumina platforms using manufacturer's instructions. The resulting DNA sequence was aligned to the reference genome reported in van Bakel et al (2011). DNA sequence variants were then determined and filtered for high quality/confidence base variants.

Over 170 plants from more than 15 accessions have been analysed. Accessions showed varying degree of diversity, including: high CBD producing plants; CBD/THC producing plants; and high THC producing plants. See FIGS. 3, 3A and 3B.

Initial genome sequencing identified >24 million variant single nucleotide polymorphisms (SNPs). >2.7 million of these provide high quality variant sites in the genome that can be utilised in the Cannabis genome.

### EXAMPLE 4—ANALYSIS OF THE THC-SYNTHASE GENE

Whole genome sequence data of the strains analysed allows the analysis of the THC-synthase gene (THCAS). The THCAS gene sequence is shown in SEQ ID NO: 1. The corresponding protein sequence is shown in SEQ ID NO: 2. Both sequences are reproduced from genbank:AB057805.

THCAS sequence [genbank:AB057805] [to query the PK genome, a single scaffold of 12.6 kb (scaffold19603, [genbank: JH239911] corresponding to SEQ ID NO: 7) was identified that contained the THCAS gene as a single 1638 bp exon with 99% nucleotide identity to the published THCAS sequence. Querying the PK transcriptome returned the same THCAS transcript (PK29242.1, [genbank:JP450547] corresponding to SEQ ID NO: 9) that was found to be expressed at high abundance in female flowers. Also there is a THCAS-like pseudogene (scaffold1330 [genbank: JH227480] corresponding to SEQ ID NO: 10, 91% nucleotide identity to THCAS)

SNP loci have been identified in the THCAS gene that alter amino acids. Plants having high CBD were found to with a single nucleic acid change resulting in amino acid change from Lysine to Methionine at base 8190 and Leucine to Phenylalanine at base 8201 in scaffold 19603. See FIG. 4.

The nucleic acid changes are shown in the 120 bp fragment of the THCAS gene of FIG. 4 also as shown in SEQ ID NO: 3.

gccggagctacccttggagaagtttattattggatcaatgag
aagaatgagaatcttagttttcctggtgggtattgccctact
gttggcgtaggtggacactttagtggaggaggctat

A nucleic acid change at position 8190 corresponds to highlighted change A to C. A nucleic acid change at position 8201 corresponds to C to T.

Without being bound by any particular theory, it is believed that the change in amino acid sequence in the THCAS may play a role in methylation patterns. This may influence the level of the cannabinoid THC in the plant that is converted from the CBGA precursor.

### EXAMPLE 5—ANALYSIS OF THE CBD-SYNTHASE GENE

Whole genome sequence data of the strains analysed allows the analysis of the CBD-synthase gene (CBDAS). The CBDAS gene sequence is shown in SEQ ID NO: 4. The corresponding protein sequence is shown in SEQ ID NO: 5. Both sequences are reproduced from genbank:AB292682.

CBDA synthase (CBDAS) sequence [genbank:AB292682] to query the PK genome as many as three scaffolds that contain CBDAS pseudogenes (scaffold39155 [genbank:AGQN01159678] corresponding to SEQ ID NO: 8, 95% nucleotide identity to CBDAS; scaffold6274 [genbank:JH231038] corresponding to SEQ ID NO: 11+scaffold74778 [genbank:JH266266] corresponding to SEQ ID NO: 12 combined, 94% identity; and scaffold99205 [genbank: AGQN01254730] corresponding to SEQ ID NO: 13, 94% identity), all of which contained premature stop codons and frameshift mutations. See, van Bakel et al. (2011).

TABLE 1

| | Bp | High CBD Strains % | | | High THC Strains % | | |
|---|---|---|---|---|---|---|---|
| Gene | position | Ref | Het | Alt | Ref | Het | Alt |
| CBDAS | 2839 | 0.00 | 0.80 | 0.20 | 0.99 | 0.01 | 0.00 |
| CBDAS | 2957 | 0.00 | 0.47 | 0.53 | 0.85 | 0.14 | 0.01 |
| CBDAS | 3223 | 0.00 | 0.90 | 0.10 | 0.99 | 0.01 | 0.00 |
| CBDAS | 3448 | 0.00 | 0.00 | 1.00 | 0.02 | 0.74 | 0.24 |

The reference genome sequence from Purple Kush (PK) contains 4 stop codons at the base positions listed in TABLE 1 above within the scaffold 39155 compared to the reference CBDAS sequence in GenBank. Table 1 details the proportion of the samples from the pan genome analysis of cannabis plants of varying chemotypic classes that contain the reference sequence allele (stop codons in this case) versus the alternative allele (Alt) (functional amino acid producing codon). Light grey shading indicates samples with 0% and dark grey shading indicates samples with >50%. No shading indicate samples between 0% and 50%. High CBD content strains do not contain any samples that are only the reference allele at any of the positions, whilst the high THC content strains, with little or no CBD production are almost exclusively containing the reference non-functional alleles at each of the 4 positions.

FIG. 5 shows analysis of CBD gene and identification of premature stop codon at position 3448 of scaffold 39155.

Without being bound by any particular theory, it is believed that the change in nucleic acid sequence at any one of these positions results in premature stop in the expression of the CBDAS gene. This may influence the level of cannabinoid CBD in the plant that is converted from the CBGA precursor.

### EXAMPLE 6—ANALYSIS OF TRICHOME DEVELOPMENT IN CANNABIS PLANT

Both cannabinoids and terpenes are manufactured in the small resin glands present on the flowers and the main fan leaves of late-stage cannabis plants called trichomes. Trichomes are microscopic, mushroom-like protrusions from the surface of the buds, fan leaves and even on the stalk of the plants. It is within the head of these protrusions where cannabinoids and terpenes are produced in the cannabis plant.

Analysis of transcriptome and metabolome in the specific resin-producing cells from the trichome is possible through cell capture laser capture micro-dissection.

### EXAMPLE 7—PLANT TISSUE CULTURE OF MEDICINAL CANNABIS

Plant tissue culture techniques have been developed to enable:
Long term maintenance of strains for stability
Transport of specific plant genetics internationally
Genome editing for the development of designer strains

See FIGS. 6 and 7.

### EXAMPLE 8—METABOLOME ANALYSIS IN MEDICINAL CANNABIS

The metabolome of medicinal cannabis has been analysed, that is an assessment of endogenous metabolites in each strain. Analytical platforms that have been used include
GCMS for volatilomics;
LCMS for in-depth metabolomics;
UHPLC-PDA quantification to meet stringent GMP requirements;
NMR for rapid non-selective metabolomics;
Production via SFE.

### EXAMPLE 9—VOLATOLOMICS ANALYSIS BY GCMS AND SPME

Terpenes or terpenoids are volatile unsaturated hydrocarbons found in plants. These are responsible for the aroma differences between cultivars. Some are bioactive and are believed to contribute to the “entourage effect”.

Air-dried and cured plant material were prepared for analysis. The air-dried buds were coarsely ground and placed into a vial for analysis. A second sample of the same material was cured (heated at 120° C. for 2 hours), cooled and placed into another vial for analysis. The material was left in each sealed vial for several hours to allow the volatiles to equilibrate between the dried material and headspace. For static headspace analysis 1 ml was sampled from the headspace of each vial. For SPME the fibre was exposed to the vial headspace for 20 sec.

FIG. 9 shows that several different compounds can be detected by GCMS and the results compared across different cultivars.

FIG. 10 shows that detection of such compounds can be enhanced with the use of SPME.

Monoterpenes (FIG. 11), sesquiterpenes (FIG. 12) and alcohols and aldehydes (FIG. 13) were detected at various levels in three different strains.

FIG. 14 shows that the detection is more readily determined in air dried samples compared to cured samples. There was a 99.5% reduction in total peak area in cured samples.

### EXAMPLE 9—LCMS FOR IN-DEPTH CHEMOTYPING

Liquid chromatography mass spectrometry (LCMS) allows the identification of cannabinoids by high resolution mass spectra and fragmentation.

FIG. 15 shows analysis of ion extracted chromatograms of mixed standards.

FIG. 16 shows the comparison of cannabinoid composition in both dried (air-dried) and cured plant material extracted with methanol prior to analysis. LCMS analysis of each sample shows that when the sample is treated at 120° C. for 2 hrs the cannabinoids are decarboxylated.

### EXAMPLE 10—UHPLC-PDA QUANTIFICATION

UHPLC-PDA (an analytical method using high performance liquid chromatography equipped with photodiode array detector) is used to quantify cannabinoids present in each sample extracts derived from specific cannabis strains. Protocols have been developed to standardise analysis methods under GMP requirements.

The protocols can be used to differentiate between strains (FIG. 17) and developmental chemotyping of strains (FIG. 18).

### EXAMPLE 11—NMR FOR RAPID METABOLOMICS AND IDENTIFICATION OF UNKNOWN/NOVEL METABOLITES

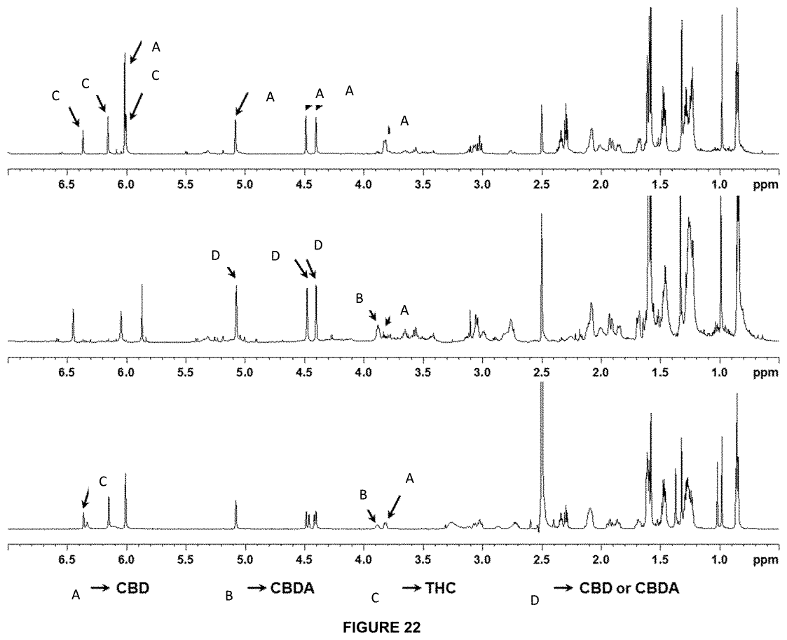

NMR spectra for cannabinoids have been determined. FIG. 19 shows ¹H NMR spectrum of CBD and CBDA. FIG. 20 shows NMR spectrum of cannabinoids. These standards can then be used to determine the composition of metaboloites in specific strains. FIGS. 21 and 22 show the NMR spectra of a cannabis plant. Cannabinoids are responsible for the dominant spectral features through other metabolites, such as glucose, are also detected.

### EXAMPLE 12—SUPER CRITICAL EXTRACTION (SFE) OF CANNABINOIDS FROM CANNABIS PLANT

SFE uses liquid carbon dioxide to extract cannabinoids from either resin or cured biomass derived from the cannabis plant. TABLE 2 below shows the design of experiment principles applied to optimise extraction of CBD and THC cannabinoids.

TABLE 2

| | CO₂ | Extraction | Extraction | Extraction | | |
|---|---|---|---|---|---|---|
| | Flowrate | time | pressure | weight | CBD in API | THC in API |
| Run | g/min | mins | bar | G | ug/g | ug/g |
| 1 | 150 | 600 | 320 | 71.0 | 113461.8 | 187567.9 |
| 2 | 40 | 600 | 150 | 27.5 | 120778.4 | 76111.6 |
| 3 | 40 | 240 | 320 | 4.2 | 133192.1 | 149470.9 |
| 4 | 40 | 240 | 150 | 9.1 | 191714.4 | 132256.5 |
| 5 | 150 | 240 | 320 | 55.1 | 137755.8 | 161929.7 |
| 6 | 40 | 600 | 320 | 55.9 | 107648.9 | 193434.9 |
| 7 | 150 | 600 | 150 | 56.3 | 150677.0 | 174808.5 |
| 8 | 150 | 240 | 150 | 50.8 | 141611.7 | 200199.2 |
| 9 | 95 | 420 | 235 | 62.7 | 105506.2 | 211542.9 |
| 10 | 95 | 420 | 235 | 57.8 | 105120.3 | 208504.9 |
| 11 | 95 | 420 | 235 | 57.2 | 103474.9 | 215808.2 |
| 12 | 150 | 600 | 320 | 68.1 | 103588.4 | 191314.2 |
| 13 | 150 | 240 | 320 | 62.7 | 103167.1 | 198966.7 |
| 14 | 150 | 600 | 150 | 58.3 | 106741.0 | 218240.3 |
| 15 | 95 | 600 | 150 | 47.7 | 132774.0 | 209962.0 |

TABLE 3 below shows the optimised extraction conditions for cannabis strains

| CO₂ | Extraction | Extraction | Extraction |
|---|---|---|---|
| Flowrate | time | pressure | weight |
| g/min | mins | bar | g |
| 150 | 390 | 150 | 80 |

Finally, it is to be understood that various alterations, modifications and/or additions may be made without departing from the spirit of the present invention as outlined herein.

### REFERENCES

Van Bakel et al “The draft genome and transcriptome of Cannabis sativa” Genome Biology (2011) 12: R102
Mandolino et al (1999) “Identification of DNA markers linked to the male sex in dioecious hemp (Cannabis sativa L.)” Theor Appl Genet 98:86-92.
Pacifico et al (2006) “Genetics and marker-assisted selection of the chemotype in Cannabis sativa L.” Molecular Breeding 17:257-268.
Shinozuka et al (2015) “A simple method for semi-random DNA amplicon fragmentation using the methylation-dependent restriction enzyme MspJI” BMC Biotechnology 15:25.

1. A method of identifying a cannabis plant having high THC content and/or high CBD content, wherein the method includes detecting a genetic variation associated with the THCAS gene and/or CBDAS gene in the cannabis plant.

2. The method according to claim 1, wherein the cannabis plant having a high THC content and/or high CBD content has one or more genetic variations associated with the THCAS gene.

3. The method according to claim 2, wherein the genetic variation is a single nucleic acid change at position 8190 in the THCAS gene within scaffold 19603 [genbank: JH239911] as shown in SEQ ID NO: 7.

4. The method according to claim 2, wherein the genetic variation is a single nucleotide change at position 8201 in the THCAS gene within scaffold 19603 [genbank: JH239911] as shown in SEQ ID NO: 7.

5. The method according to claim 1, wherein the cannabis plant having a high THC content and/or high CBD content has one or more genetic variations associated with the CBDAS gene.

6. The method according to claim 5, wherein the genetic variation is a single nucleotide change at position 2839 in the CBDAS gene within scaffold 39155 [genbank: AGQN01159678] as shown in SEQ ID NO: 8.

7. The method according to claim 5, wherein the genetic variation is a single nucleotide change at position 2957 in the CBDAS gene within scaffold 39155 [genbank: AGQN01159678] as shown in SEQ ID NO: 8.

8. The method according to claim 5, wherein the genetic variation is a single nucleotide change at position 3223 in the CBDAS gene within scaffold 39155 [genbank: AGQN01159678] as shown in SEQ ID NO: 8.

9. The method according to claim 5, wherein the genetic variation is a single nucleotide change at position 3448 in the CBDAS gene within scaffold 39155 [genbank: AGQN01159678] as shown in SEQ ID NO: 8.

10. The method according to claim 1, wherein the cannabis plant is selected from the species or hybrids of Cannabis sativa, Cannabis indica, and Cannabis ruderalis.

11. The method according to claim 10, wherein the cannabis plant is Cannabis sativa.

12. The method according to claim 1, wherein the cannabis plant having a high THC content contains a ratio by weight of THC to CBD of more than about 1, preferably more than about 1.2, more preferably more than about 1.5, more preferably more than about 2.

13. The method according to claim 1, wherein the cannabis plant having a high THC content contains a ratio by weight of THC to CBD of between about 400:1 and 2:1, preferably about 100:1 to 2:1, more preferably about 50:1 to 2:1, more preferably about 25:1 to 2:1, more preferably about 10:1 to 2:1, more preferably about 5:1 to 2:1.

14. The method according to claim 1, wherein the cannabis plant having a high CBD content contains a ratio by weight of CBD to THC of more than about 1, preferably more than about 1.2, more preferably more than about 1.5, more preferably more than about 2.

15. The method according to claim 1, wherein the cannabis plant having a high CBD content contains a ratio by weight of CBD to THC of between about 400:1 and 2:1, preferably about 100:1 to 2:1, more preferably about 50:1 to 2:1, more preferably about 25:1 to 2:1, more preferably about 10:1 to 2:1, more preferably about 5:1 to 2:1.

16. A cannabis plant having a high THC content and/or high CBD content identified according to the method of claim 1.

17. A seed, cell, part of a plant and/or a plant-derived product derived from a cannabis plant according to claim 16.

18. (canceled)

19. A method of preparing a pharmaceutical composition comprising:
(a) providing a cannabis plant according to claim 16 or a seed, cell, part of a plant and/or a plant-derived product according to claim 17; and
(b) preparing an extract of (a).

20. The method according to claim 19, further comprising:
heating plant material of (a) to a temperature of from about 60° C. to about 225° C., preferably about 100° C. to about 150° C., more preferably about 110° C. to 130° C., more preferably at about 120° C., to decarboxylate the acid form of any cannabinoids present in the extract.

21. The method according to claim 19, further comprising:
preparing the extract by one selected from the group consisting of maceration, percolation, extraction with a solvent and supercritical fluid extraction.

22. A pharmaceutical composition prepared by the method according to claim 19.

23. The pharmaceutical composition according to claim 22, further comprising one or more other cannabinoids selected from: cannabigerol (CBG), cannabichromene (CBC), cannabidiol (CBD), tetrandrocannabinol (THC), cannabinol (CBN), cannabinodiol (CBDL), cannabicyclol (CBL), tetrahydrocannabivarin (THCV), cannabidivarin (CBDV), cannabichromevarian (CBCV), cannabigerovarin (CBGV), cannabigerol monomethyl ether (CBGM), cannabinerolic acid, cannabidiolic acid JCBDA), cannabinol propyl variant (CBNV), cannabitriol (CBO), tetrahydrocannabinolic acid (THCA), tetrahydrocannabivarinic acid (THCVA), d9-THC, exo-THC, 11-OH-d9-THC, 11-nor-d9-THC, d9-THCA-A, and d8-THC12, preferably CBDA and THCA.

24. The pharmaceutical composition according to claim 23, wherein the composition further comprises one or more terpenes selected from the group consisting of aromadendrene, bergamottin, bergamotol, bisabolene, borneol, alpha-3-carene, caryophyllene, cinole/eucalyptol, p-cymene, dihydrojasmone, elemene, farnesene, fenchol, geranylacetate, guaiol, humulene, isopulegol, limonene, linalool, menthone, menthol, menthofuran, myrcene, nerylacetate, neomenthylacetate, ocimene, perillylalcohol, phellandrene, pinene, pulegone, sabinene, terpinene, terpinol, and terpineol-4-ol, terpinolene, and derivatives, isomers, and enantiomers thereof.

25. The pharmaceutical composition according to claim 22 for use in the manufacture of a medicament for the treatment of pain and/or management thereof or epilepsy.

26. The pharmaceutical composition according to claim 25 having CBD in an amount by weight greater than the amount by weight of THC for use in the treatment of epilepsy.

27. The pharmaceutical composition according to claim 26 having THC in an amount by weight greater than the amount by weight of CBD for use in the treatment of pain and/or management thereof.

28. A method of breeding a cannabis plant comprising:
identifying or selecting a cannabis plant having high THC content and/or high CBD content according to the method of claim 1.

29. (canceled)


SEQUENCE LISTING (6 sequences)

SEQ ID NO: 1 - DNA, 1885 bp, Cannabis sativa
aaaaaaatcattaggactgaagaaaaatgaattgctcagcattttccttttggtttgtttgcaaaataatatttttctttctctcattccatatccaaatttcaatagctaatcctcgagaaaacttccttaaatgcttctcaaaacatattcccaacaatgtagcaaatccaaaactcgtatacactcaacacgaccaattgtatatgtctatcctgaattcgacaatacaaaatcttagattcatctctgatacaaccccaaaaccactcgttattgtcactccttcaaataactcccatatccaagcaactattttatgctctaagaaagttggcttgcagattcgaactcgaagcggtggccatgatgctgagggtatgtcctacatatctcaagtcccatttgttgtagtagacttgagaaacatgcattcgatcaaaatagatgttcatagccaaactgcgtgggttgaagccggagctacccttggagaagtttattattggatcaatgagaagaatgagaatcttagttttcctggtgggtattgccctactgttggcgtaggtggacactttagtggaggaggctatggagcattgatgcgaaattatggccttgcggctgataatattattgatgcacacttagtcaatgttgatggaaaagttctagatcgaaaatccatgggagaagatctgttttgggctatacgtggtggtggaggagaaaactttggaatcattgcagcatggaaaatcaaactggttgctgtcccatcaaagtctactatattcagtgttaaaaagaacatggagatacatgggcttgtcaagttatttaacaaatggcaaaatattgcttacaagtatgacaaagatttagtactcatgactcacttcataacaaagaatattacagataatcatgggaagaataagactacagtacatggttacttctcttcaatttttcatggtggagtggatagtctagtcgacttgatgaacaagagctttcctgagttgggtattaaaaaaactgattgcaaagaatttagctggattgatacaaccatcttctacagtggtgttgtaaattttaacactgctaattttaaaaaggaaattttgcttgatagatcagctgggaagaagacggctttctcaattaagttagactatgttaagaaaccaattccagaaactgcaatggtcaaaattttggaaaaattatatgaagaagatgtaggagctgggatgtatgtgttgtacccttacggtggtataatggaggagatttcagaatcagcaattccattccctcatcgagctggaataatgtatgaactttggtacactgcttcctgggagaagcaagaagataatgaaaagcatataaactgggttcgaagtgtttataattttacgactccttatgtgtcccaaaatccaagattggcgtatctcaattatagggaccttgatttaggaaaaactaatcatgcgagtcctaataattacacacaagcacgtatttggggtgaaaagtattttggtaaaaattttaacaggttagttaaggtgaaaactaaagttgatcccaataatttttttagaaacgaacaaagtatcccacctcttccaccgcatcatcattaattatctttaaatagatatatttcccttatcaattagttaatcattataccatacatacatttattgtatatagtttatctactcatattatgtatgctcccaagtatgaaaatctacattagaactgtgtagacaatcataagatatatttaataaaataaattgtctttcttatttcaatagcaaataaaataatattattttaaaaaaaaaaaaaaaaa

SEQ ID NO: 2 - protein, 545 aa, Cannabis sativa
MNCSAFSFWFVCKIIFFFLSFHIQISIANPRENFLKCFSKHIPNNVANPKLVYTQHDQLYMSILNSTIQNLRFISDTTPKPLVIVTPSNNSHIQATILCSKKVGLQIRTRSGGHDAEGMSYISQVPFVVVDLRNMHSIKIDVHSQTAWVEAGATLGEVYYWINEKNENLSFPGGYCPTVGVGGHFSGGGYGALMRNYGLAADNIIDAHLVNVDGKVLDRKSMGEDLFWAIRGGGGENFGIIAAWKIKLVAVPSKSTIFSVKKNMEIHGLVKLFNKWQNIAYKYDKDLVLMTHFITKNITDNHGKNKTTVHGYFSSIFHGGVDSLVDLMNKSFPELGIKKTDCKEFSWIDTTIFYSGVVNFNTANFKKEILLDRSAGKKTAFSIKLDYVKKPIPETAMVKILEKLYEEDVGAGMYVLYPYGGIMEEISESAIPFPHRAGIMYELWYTASWEKQEDNEKHINWVRSVYNFTTPYVSQNPRLAYLNYRDLDLGKTNHASPNNYTQARIWGEKYFGKNFNRLVKVKTKVDPNNFFRNEQSIPPLPPHHH

SEQ ID NO: 3 - DNA, 120 bp, Cannabis sativa
gccggagctacccttggagaagtttattattggatcaatgagaagaatgagaatcttagttttcctggtgggtattgccctactgttggcgtaggtggacactttagtggaggaggctat

SEQ ID NO: 4 - DNA, 1635 bp, Cannabis Sativa
atgaagtgctcaacattctccttttggtttgtttgcaagataatatttttctttttctcattcaatatccaaacttccattgctaatcctcgagaaaacttccttaaatgcttctcgcaatatattcccaataatgcaacaaatctaaaactcgtatacactcaaaacaacccattgtatatgtctgtcctaaattcgacaatacacaatcttagattcacctctgacacaaccccaaaaccacttgttatcgtcactccttcacatgtctctcatatccaaggcactattctatgctccaagaaagttggcttgcagattcgaactcgaagtggtggtcatgattctgagggcatgtcctacatatctcaagtcccatttgttatagtagacttgagaaacatgcgttcaatcaaaatagatgttcatagccaaactgcatgggttgaagccggagctacccttggagaagtttattattgggttaatgagaaaaatgagaatcttagtttggcggctgggtattgccctactgtttgcgcaggtggacactttggtggaggaggctatggaccattgatgagaaactatggcctcgcggctgataatatcattgatgcacacttagtcaacgttcatggaaaagtgctagatcgaaaatctatgggggaagatctcttttgggctttacgtggtggtggagcagaaagcttcggaatcattgtagcatggaaaattagactggttgctgtcccaaagtctactatgtttagtgttaaaaagatcatggagatacatgagcttgtcaagttagttaacaaatggcaaaatattgcttacaagtatgacaaagatttattactcatgactcacttcataactaggaacattacagataatcaagggaagaataagacagcaatacacacttacttctcttcagttttccttggtggagtggatagtctagtcgacttgatgaacaagagttttcctgagttgggtattaaaaaaacggattgcagacaattgagctggattgatactatcatcttctatagtggtgttgtaaattacgacactgataattttaacaaggaaattttgcttgatagatccgctgggcagaacggtgctttcaagattaagttagactacgttaagaaaccaattccagaatctgtatttgtccaaattttggaaaaattatatgaagaagatataggagctgggatgtatgcgttgtacccttacggtggtataatggatgagatttcagaatcagcaattccattccctcatcgagctggaatcttgtatgagttatggtacatatgtagttgggagaagcaagaagataacgaaaagcatctaaactggattagaaatatttataacttcatgactccttatgtgtccaaaaatccaagattggcatatctcaattatagagaccttgatataggaataaatgatcccaagaatccaaataattacacacaagcacgtatttggggtgagaagtattttggtaaaaattttgacaggctagtaaaagtgaaaaccctggttgatcccaataacttttttagaaacgaacaaagcatcccacctcttccacggcatcgtcattaa

SEQ ID NO: 5 - protein, 544 aa, Cannabis sativa
MKCSTFSFWFVCKIIFFFFSFNIQTSIANPRENFLKCFSQYIPNNATNLKLVYTQNNPLYMSVLNSTIHNLRFTSDTTPKPLVIVTPSHVSHIQGTILCSKKVGLQIRTRSGGHDSEGMSYISQVPFVIVDLRNMRSIKIDVHSQTAWVEAGATLGEVYYWVNEKNENLSLAAGYCPTVCAGGHFGGGGYGPLMRNYGLAADNIIDAHLVNVHGKVLDRKSMGEDLFWALRGGGAESFGIIVAWKIRLVAVPKSTMFSVKKIMEIHELVKLVNKWQNIAYKYDKDLLLMTHFITRNITDNQGKNKTAIHTYFSSVFLGGVDSLVDLMNKSFPELGIKKTDCRQLSWIDTIIFYSGVVNYDTDNFNKEILLDRSAGQNGAFKIKLDYVKKPIPESVFVQILEKLYEEDIGAGMYALYPYGGIMDEISESAIPFPHRAGILYELWYICSWEKQEDNEKHLNWIRNIYNFMTPYVSKNPRLAYLNYRDLDIGINDPKNPNNYTQARIWGEKYFGKNFDRLVKVKTLVDPNNFFRNEQSIPPLPRHRH

SEQ ID NO: 6 - DNA, 117 bp, Cannabis sativa
attcctagttatgaagtgagtcatgagtaataaatctttgtcatacttgtaagcaatattttgccatttgttaactcacttgacaagctcatgtatctccatgtctttttaacacta